(19) United States
(12) Patent Application Publication
(10) Pub. No.: US 2012/0277419 A1
(43) Pub. Date: Nov. 1, 2012

(54) METHOD FOR MANUFACTURING PEGYLATED OLIGONUCLEOTIDES

(76) Inventors: DOUGLAS BROOKS, DURHAM, NC (US); CHRISTOPHER P. RUSCONI, DURHAM, NC (US)

(21) Appl. No.: 13/457,095

(22) Filed: Apr. 26, 2012

Related U.S. Application Data
(60) Provisional application No. 61/479,226, filed on Apr. 26, 2011.

(51) Int. Cl. C07H 21/00

(52) U.S. Cl. 536/25.3

(57) ABSTRACT

A method for preparing a therapeutic pegylated oligonucleotide is described. The method involves synthesis, cleavage and purification steps designed to improve the efficiency whereby the therapeutic oligonucleotide may be prepared. Also described are methods for large scale preparation at the improved efficiency.

### CROSS-REFERENCE TO RELATED APPLICATIONS

This application claims the benefit of U.S. Provisional Application Ser. No. 61/479,226, filed on Apr. 26, 2011, which is hereby incorporated by reference in its entirety.

### BACKGROUND

Nucleic acids have conventionally been thought of as primarily playing an informational role in biological processes. In the past decades it has become clear that the three dimensional structure of nucleic acids can give them the capacity to interact with and regulate proteins. Such nucleic acid ligands or “aptamers” are short DNA or RNA oligomers which can bind to a given ligand with high affinity and specificity. As a class, the three dimensional structures of aptamers are sufficiently variable to allow aptamers to bind to and act as ligands for virtually any chemical compound, whether monomeric or polymeric. Aptamers have emerged as promising new diagnostic and therapeutic compounds, particularly in cancer therapy and the regulation of blood coagulation.

A number of third parties have applied for and secured patents covering the identification, manufacture and use of aptamers. Gold and Tuerk are generally credited with first developing the SELEX method for isolating aptamers, and their method is described in a number of United States patents including U.S. Pat. Nos. 5,670,637, 5,696,249, 5,843,653, 6,110,900, and 5,270,163. Thomas Bruice et al. reported a process for producing aptamers in U.S. Pat. No. 5,686,242, which differs from the original SELEX process reported by Tuerk and Gold because it employs strictly random oligonucleotides during the screening sequence. The oligonucleotides screened in the '242 patent lack the oligonucleotide primers that are present in oligonucleotides screened in the SELEX process.

Sullenger, Rusconi, Kontos and White in WO 02/26932 describe RNA aptamers that bind to coagulation factors, E2F family transcription factors, Ang1, Ang2, and fragments or peptides thereof, transcription factors, autoimmune antibodies and cell surface receptors useful in the modulation of hemostasis and other biologic events. See also Rusconi et al, Thrombosis and Haemostasis 83:841-848 (2000), White et al, J. Clin Invest 106:929-34 (2000), Ishizaki et al, Nat Med 2:1386-1389 (1996), and Lee et al, Nat. Biotechnol. 15:41-45 (1997)).

In order for an aptamer to be suitable for use as a therapeutic, it is preferably inexpensive to synthesize, safe and stable in vivo. RNA and DNA aptamers composed of all ribose or deoxyribose nucleotides with no modifications to the phosphodiester backbone are typically not stable in vivo because of their susceptibility to degradation by nucleases. Resistance to nuclease degradation can be greatly increased by the incorporation of modifying groups at the 2′-position.

In addition to clearance by nucleases, oligonucleotide therapeutics are subject to elimination via renal filtration. As such, a nuclease-resistant oligonucleotide administered intravenously typically exhibits an in vivo half-life of <10 min, unless filtration can be blocked. This can be accomplished by either facilitating rapid distribution out of the blood stream into tissues or by increasing the apparent molecular weight of the oligonucleotide above the effective size cut-off for the glomerulus. Conjugation of small therapeutics to a polyalkylene oxide polymer (e.g., PEGylation) can dramatically lengthen residence times of aptamers in circulation, thereby decreasing dosing frequency and enhancing effectiveness against vascular targets.

The large-scale manufacture of pegylated oligonucleotides which meet the standards set by the Food and Drug Administration (FDA) and other international regulatory agencies involves numerous steps for the synthesis, purification and characterization of the final product administered to the patient. Each step costs significant amounts of both time and money. Accordingly, there is a need to optimize efficient and feasible manufacturing methods for this class of therapeutics.

### BRIEF SUMMARY

In one aspect, a method of preparing a polyalkylene oxide (PAO)-conjugated oligonucleotide is provided. In one embodiment, the oligonucleotide is an RNA aptamer, wherein the aptamer has a secondary structure. In yet another embodiment, the oligonucleotide is a neutralization agent, or active control agent of an aptamer.

In one embodiment, the oligonucleotide is synthesized using solid phase synthesis. In another embodiment, the oligonucleotide is synthesized using at least one modified nucleotide.

In one embodiment, the method of manufacturing the PAO-conjugated oligonucleotide comprises: synthesizing a non-pegylated oligonucleotide on a solid support, cleaving the non-pegylated oligonucleotide from the solid support and deprotecting the non-pegylated oligonucleotide, desalting the non-pegylated oligonucleotide, pegylating the non-pegylated oligonucleotide to produce a pegylated oligonucleotide, purifying the pegylated oligonucleotide, and desalting the pegylated oligonucleotide. In one embodiment, the desalting the non-pegylated oligonucleotide comprises ultrafiltration.

In one embodiment, the method of manufacturing the PAO-conjugated oligonucleotide comprises: synthesizing a non-pegylated oligonucleotide on a solid support, cleaving the non-pegylated oligonucleotide from the solid support and deprotecting the non-pegylated oligonucleotide, salt-exchanging the non-pegylated oligonucleotide, pegylating the non-pegylated oligonucleotide to produce a pegylated oligonucleotide, purifying the pegylated oligonucleotide, and desalting the pegylated oligonucleotide. In one embodiment, the salt-exchanging the non-pegylated oligonucleotide comprises ultrafiltration.

In one embodiment, the method of manufacturing the PAO-conjugated oligonucleotide comprises: synthesizing a non-pegylated oligonucleotide on a solid support, cleaving the non-pegylated oligonucleotide from the solid support and deprotecting the non-pegylated oligonucleotide, desalting and salt-exchanging the non-pegylated oligonucleotide, pegylating the non-pegylated oligonucleotide to produce a pegylated oligonucleotide, purifying the pegylated oligonucleotide, and desalting the pegylated oligonucleotide. In one embodiment, the desalting and salt-exchanging the non-pegylated oligonucleotide comprises ultrafiltration.

In one embodiment, the method further comprises freeze drying the pegylated oligonucleotide. In another embodiment, the freeze-drying step is performed after the step of desalting and further purifying the pegylated oligonucleotide using ultrafiltration.

In another embodiment, the purifying the pegylated oligonucleotide comprises using ultrafiltration with an ultrafiltration membrane with a molecular weight cutoff less than the molecular weight of the pegylated oligonucleotide. In yet another embodiment, the ultrafiltration membrane has a molecular weight cutoff of about 10 kD, 20 kD or 30 kD. In still another embodiment, the ultrafiltration membrane has a molecular weight cutoff of about 10 kD to about 20 kD or of about 20 kD to about 30 kD.

In one embodiment, the method does not involve an ion-exchange purification of the non-pegylated oligonucleotide after cleaving the non-pegylated oligonucleotide from the solid support and deprotecting the non-pegylated oligonucleotide. In another embodiment, the method does not involve an ion-exchange purification of the non-pegylated oligonucleotide before desalting and/or salt-exchanging the non-pegylated oligonucleotide using ultrafiltration.

In one embodiment, the purifying the pegylated oligonucleotide comprises using anion exchange high performance liquid chromatography (HPLC).

In one embodiment, the non-pegylated oligonucleotide comprises at least one modified base moiety.

In another embodiment, the at least one modified base moiety is selected from the group consisting of 5-fluorouracil, 5-fluorocytosine, 5-bromouracil, 5-bromocytosine, 5-chlorouracil, 5-chlorocytosine, 5-iodouracil, 5-iodocytosine, 5-methylcytosine, 5-methyluracil, hypoxanthine, xanthine, 4-acetylcytosine, 5-(carboxyhydroxymethyl)uracil, 5-carboxymethylamin-O-methyl thiouridine, 5-carboxymethylamin-O-methyluracil, dihydrouracil, beta-D-galactosylqueosine, inosine, N6-isopentenyladenine, 1-methylguanine, 1-methylinosine, 2,2-dimethylguanine,2-methyladenine, 2-methylguanine, 3-methylcytosine, 6-methylcytosine, N6-adenine, 7-methylguanine, 5-methylamin-O-methyluracil, 5-methoxyamin-O-methyl-2-thiouracil, beta-D-mannosylqueosine, 5′-methoxycarboxymethyluracil, 5-methoxyuracil, 5-methoxycytosine, 2-methylthio-N6-isopentenyladenine, uracil oxyacetic acid (v), butoxosine, pseudouracil, queosine, 2-thiocytosine, 5-methyl thiouracil, 2-thiouracil, 4-thiouracil, 5-methyluracil, uracil-5-oxyacetic acid methylester, uracil oxyacetic acid (v), 5-methyl thiouracil, 3-(3-amino-3-N carboxypropyl)uracil, (acp3)w, and 2,6-diaminopurine.

In one embodiment, the non-pegylated oligonucleotide includes one or more 2′-O-methyl modified nucleotides. In another embodiment, the non-pegylated oligonucleotide contains one or more 2′-O-methyl and one or more 2′-fluoro modifications.

In one embodiment, the non-pegylated oligonucleotide has a secondary structure. In another embodiment, the secondary structure comprises at least one stem and at least one loop. In another embodiment, the secondary structure comprises two stems and three loops.

In one embodiment, the non-pegylated oligonucleotide comprises one or more 2′-O-methyl and/or one or more 2′-fluoro modifications. In another embodiment, the non-pegylated oligonucleotide comprises one or more 2′-O-methyl and/or one or more 2′-fluoro modifications in at least one stem and/or the at least one loop.

In one embodiment, at least one guanine in the at least one stem of the non-pegylated oligonucleotide includes a hydroxyl sugar (2′-OH). In another embodiment, the at least one uridine in the at least one stem of the non-pegylated oligonucleotide is modified with either a 2′-fluoro or 2′-O-methyl. In another embodiment, at least one cytidine in the at least one stem of the non-pegylated oligonucleotide is 2′-fluoro modified.

In one embodiment, the non-pegylated oligonucleotide comprises a spacer. In another embodiment, the spacer is a glycol spacer.

In one embodiment, the non-pegylated oligonucleotide comprises one of SEQ ID NOs:1-20. In another embodiment, the non-pegylated oligonucleotide consists one of SEQ ID NOs:1-20. In still another embodiment, the non-pegylated oligonucleotide comprises one of the modified oligonucleotides as described in Tables 1 and 2 of this specification. In yet another embodiment, the non-pegylated oligonucleotide consists of one of the modified oligonucleotides as described in Tables 1 and 2 of this specification.

In one embodiment, the non-pegylated oligonucleotide is coupled to a linker prior to conjugation to the PAO to produce a linker-conjugated non-pegylated oligonucleotide. In one embodiment, the non-pegylated oligonucleotide comprises a linker having a reactive amino group and the polyalkylene oxide is functionalized with an activated ester group. In another embodiment, the non-pegylated oligonucleotide comprises a linker having a reactive thio group and the polyalkylene oxide is functionalized with a maleimide group. In one embodiment, the activated ester group is NHS.

In one embodiment, the polyalkylene oxide is not activated. In another embodiment, the polyalkylene oxide further comprises a carboxylic acid moiety. In still another embodiment the conjugation of the PAO to the linker-conjugated non-pegylated oligonucleotide is accomplished in situ by inclusion of a water soluble coupling agent in the conjugation reaction. In one embodiment, the water soluble coupling agent is dicyclohexylcarbodiimide (DCC). In another embodiment, the water soluble coupling agent is 1-Ethyl-3-(3-dimethylaminopropyl)carbodiimide hydrochloride (EDAC).

In one embodiment, the linker-containing non-pegylated oligonucleotide is produced by conjugating or otherwise attaching a linker precursor to the oligonucleotide. In another embodiment, the linker precursor is selected from the group consisting of: a 6-(trifluoroacetamido)hexanol (2-cyanoethyl-N,N-diisopropyl)phosphoramidite, a TFA-amino C4 CED phosphoramidite, a 5′-amino modifier C3 TFA, a 5′-amino modifier 5,5′-amino modifier C12, and a 5′ thiol-modifier C6 (linkers illustrated below).

In one embodiment, the linker is a hexylamino linker.

In one embodiment, the polyalkylene oxide is polyethylene glycol (PEG). In still another embodiment, the PEG has a molecular weight of about 20 kD to about 60 kD, about 20 kD, about 30 kD, about 40 kD, about 50 kD or about 60 kD.

In one embodiment, the method of manufacturing the PAO-conjugated oligonucleotide further comprises ultrafiltration of the linker-containing oligonucleotide. In another embodiment, the ultrafiltration of the non-pegylated oligonucleotide is diafiltration against a salt solution. In yet another embodiment, the salt is a sodium or a potassium salt.

In one embodiment, ultrafiltration is performed in the presence of purified water. In another embodiment, ultrafiltration is performed in the presence of purified water following ultrafiltration in the presence of a salt solution. In one embodiment the salt is a monovalent salt.

In another embodiment, the salt is a sodium, potassium or lithium salt. In still another embodiment, the salt is sodium, potassium, or lithium of one of the following anions: Cl⁻, HSO₃⁻, BrO₃⁻, Br⁻, NO₃⁻, ClO₃⁻, HSO₄⁻, HCO₃⁻, IO₃⁻, HPO₄²⁻, formate, acetate or propionate. In yet another embodiment, the salt is sodium chloride.

In one embodiment, the final permeate has a conductivity greater than 0 μS/cm and less than about 75 μS/cm, less than about 50 μS/cm or less than about 40 μS/cm, or ranging from about 20 μS/cm to about 70 μS/cm, about 20 μS/cm to about 50 μS/cm or about 20 μS/cm to about 40 μS/cm.

In one embodiment, the osmolality of the final permeate is greater than 0 mOsm and less than or equal to about 4 mOsm, less than or equal to about 2 mOsm, less than or equal to about 1 mOsm. In another embodiment, the osmolality of the final permeate ranges from about 0.001 mOsm to about 1.0 mOsm, from about 0.5 mOsm to about 2.0 mOsm, or from about 0.5 mOsm to about 4.0 mOsm.

In one embodiment, the ultrafiltration is performed at a temperature of from 0° C. to about 50° C., or at ambient temperature, such as from about 15° C. to about 30° C.

In one embodiment, the ultrafiltration of the linker-containing oligonucleotide is performed using a membrane having a molecular weight cutoff (MWCO) of about 1 kD to about 10 kD. In yet another embodiment, the ultrafiltration is performed using a 1 kD membrane, a 2 kD membrane, a 3 kD membrane, a 5 kD membrane, an 8 kD membrane or a 10 kD membrane. In another embodiment, the ultrafiltration is performed using a membrane with a molecular weight cutoff of about 1 kD to about 5 kD or from about 3 kD to about 5 kD. In still another embodiment, the ultrafiltration membrane would have a molecular weight cutoff which is less than the molecular weight of the linker-containing oligonucleotide.

In one embodiment, the final conductivity of the final permeate is less than or equal to about 50 μS/cm. In another embodiment, the final osmolality of the final permeate if less than or equal to about 1.0 mOsm.

In one embodiment, concentration of the non-pegylated oligonucleotide is performed by ultrafiltration of the linker-containing non-pegylated oligonucleotide. In a further embodiment, concentration of the non-pegylated oligonucleotide is performed by distillation.

In one embodiment, the method of manufacturing the PAO-conjugated oligonucleotide further comprises conjugating a polyalkylene glycol precursor moiety to the linker of the linker-conjugated non-pegylated oligonucleotide. In another embodiment, the polyalkylene glycol precursor is polyethylene glycol.

In one embodiment, the method of manufacturing the PAO-conjugated oligonucleotide further comprises purification of the PAO-conjugated oligonucleotide by ion exchange chromatography. In one embodiment the ion exchange chromatography is anion exchange HPLC.

In one embodiment, the method of manufacturing the PAO-conjugated oligonucleotide further comprises purification by ultrafiltration of the eluent from the ion exchange chromatography. In one embodiment, the method of manufacturing the PAO-conjugated oligonucleotide further comprises concentration by ultrafiltration. In one embodiment, the method of manufacturing the PAO-conjugated oligonucleotide comprises purification by ultrafiltration of the pegylation mixture without anion exchange purification prior to the purification by ultrafiltration.

In one embodiment, the PAO-conjugated oligonucleotide is concentrated by distillation.

In one embodiment, the method of manufacturing the PAO-conjugated oligonucleotide further comprises freeze drying the PAO-conjugated oligonucleotide.

### DRAWINGS

FIG. 1 illustrates a process for synthesis and purification of an oligonucleotide composition.

FIGS. 2A and 2B illustrate conjugation of an oligonucleotide to a PEG moiety via a linker moiety.

FIGS. 3A-3C illustrate the structure of RB006 and RB007 and the complex formed by them.

FIGS. 4A-4B illustrates some embodiments of Process 1 and Process 2 for synthesis and purification of an oligonucleotide composition.

FIG. 5 illustrates structures of some impurities observed in the processes described herein.

FIG. 6 illustrates some embodiments of Process 1 and Process 2 for synthesis and purification of an oligonucleotide composition.

FIG. 7 shows a spectrum of an anion exchange HPLC analysis performed to analyze a pegylation reaction mixture.

FIG. 8 shows a spectrum of an anion exchange HPLC analysis performed to analyze a retentate following ultrafiltration of a pegylation reaction mixture.

FIG. 9 shows a spectrum of an anion exchange HPLC analysis performed to analyze a permeate following ultrafiltration of a pegylation reaction mixture.

FIG. 10 shows a spectrum of an ion pair HPLC analysis performed to analyze a retentate following ultrafiltration of a pegylation reaction mixture.

### DETAILED DESCRIPTION

In order to produce sufficient quantities of drug needed to support late stage clinical trials and subsequent commercial marketing, extensive efforts are made to enhance robustness and yield of the manufacturing process while maintaining product quality. In addition, there must be stringent control of key raw materials to assure better control of the overall process and quality.

Throughout the manufacturing process, products at various steps are characterized in detail to assure the necessary quality and quantity throughout the process. As a result of this characterization, it becomes possible to identify steps of the process which may benefit from further development and optimizations.

Disclosed herein is the development of an improved and more efficient method for manufacturing pegylated oligonucleotide aptamers. It was surprisingly discovered that omission of anion exchange chromatography for removal of oligonucleotide impurities prior to performing the pegylation reaction yielded a final substance indistinguishable from that produced when anion exchange purification was performed prior to the pegylation reaction. Also unexpected was that the efficiency of the pegylation of the linker-containing oligonucleotide was maintained in the process when the anion exchange purification step prior to the pegylation reaction was omitted from the process, and the pegylation reaction was conducted with a non-purified mix of linker-containing and non-linker containing oligonucleotides. Moreover, it was surprisingly discovered that ultrafiltration could efficiently remove non-pegylated oligonucleotide species that co-elute in anion exchange purification with the pegylated oligonucleotide product.

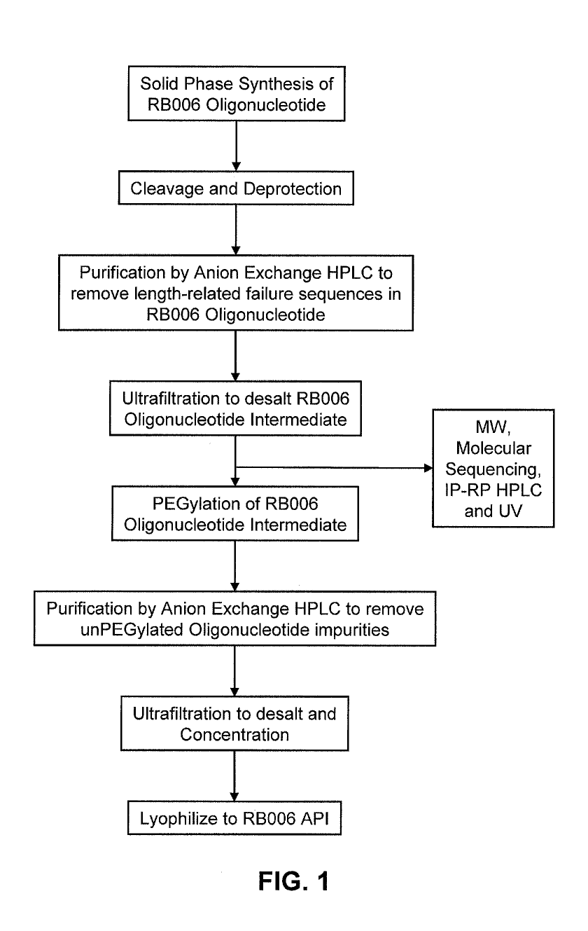

Manufacture of a therapeutic oligonucleotide is a multistep process involving solid phase chemical synthesis of the oligonucleotide strand, cleavage and deprotection of the crude oligonucleotide, purification by preparative anion exchange chromatography, desalting followed by PEGylation, purification of the pegylated oligonucleotide by preparative anion exchange chromatography to remove unpegylated oligonucleotide impurities and non-reacted PAO, ultrafiltration for desalting, and concentration and lyophilization of the final product. The entire process is schematically shown in the process flow diagram in FIG. 1.

Chemical synthesis of an oligonucleotide can be done, for example, via phosphoramidite or phosphorothioate chemistry as is well known in the art. The synthesis involves sequential coupling of activated monomers to an elongating polymer, one terminus of which is covalently attached to a solid support matrix. The solid phase approach allows for easy purification of the reaction product at each step in the synthesis by simple solvent washing of the solid phase. The oligonucleotides are sequentially assembled from the 3′-end towards the 5′-end by deprotecting the 5′-end of the support-bound molecule, allowing the support-bound molecule to react with an incoming tetrazole-activated phosphoramidite monomer, oxidizing the resulting phosphite triester to a phosphate triester, and blocking any unreacted hydroxyl groups by acetylation (capping) to prevent non-sequential coupling with the next incoming monomer to form a “deletion sequence.” This sequence of steps is repeated for subsequent coupling reactions until the full-length oligonucleotides are synthesized.

For the production of therapeutic oligonucleotides which possess increased stability in vivo, the oligonucleotides are synthesized using a variety of modifications known to those with ordinary skill in the art. For example, U.S. Pat. Nos. 5,670,633 and 6,005,087 to Cook et al. describe thermally stable 2′-fluoro oligonucleotides that are complementary to an RNA or DNA base sequence. U.S. Pat. Nos. 6,222,025 and 5,760,202 to Cook et al. describe the synthesis of 2′-0 substituted pyrimidines and oligomers containing the modified pyrimidines. Additional descriptions are found in U.S. Pat. No. 7,531,524, the contents of which are incorporated by reference in their entirety.

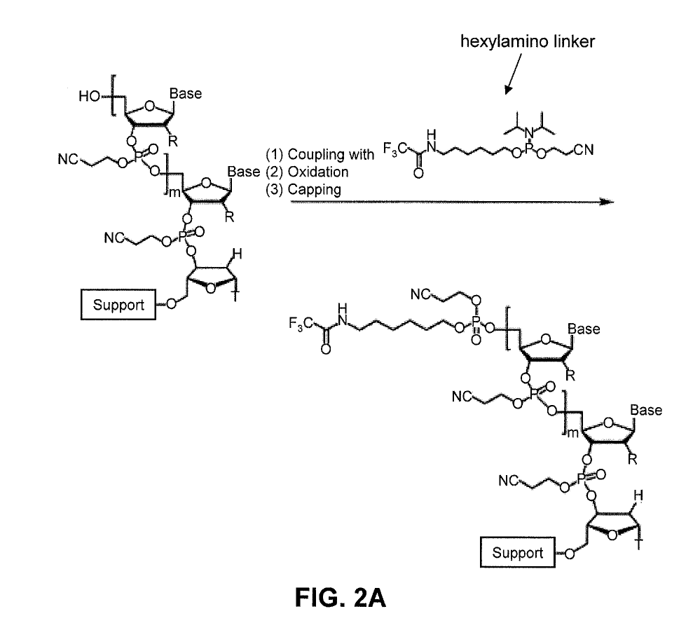

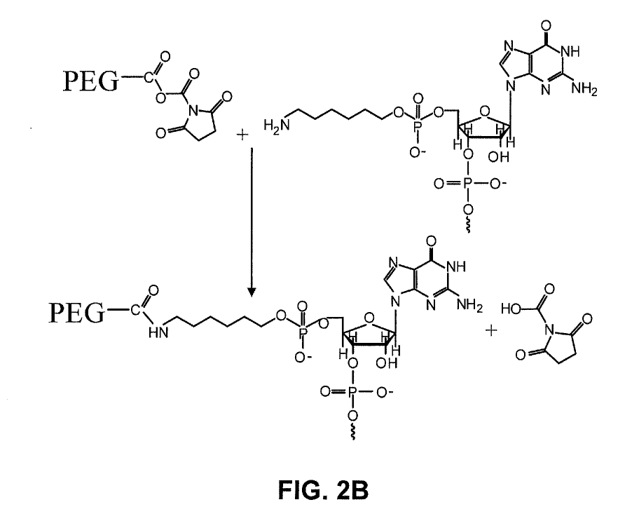

To allow subsequent conjugation to a carrier moiety, such as a polyalkylene oxide molecule, synthesis of the oligonucleotide may be completed with the addition of an appropriate linker moiety. For example, an amino linker, such as the C 6 hexylamino linker shown in FIG. 2, may be added to the 5′ end of the synthesized oligonucleotide. Other linkers that may be used are described below and include but are not limited to:

6-(trifluoroacetamido)hexanol(2-cyanoethyl-N,N-diisopropyl)phosphoramidite of the structure:

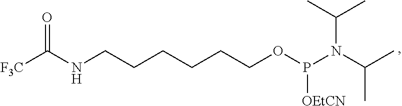

TFA-amino C4 CED phosphoramidite of the structure:

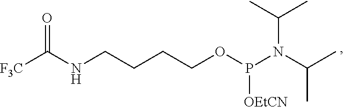

5′-amino modifier C3 TFA of the structure:

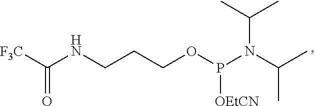

5′ amino modifier 5 of the structure:

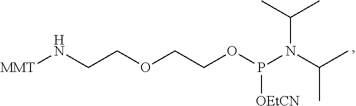

5′ amino modifier C12 of the structure:

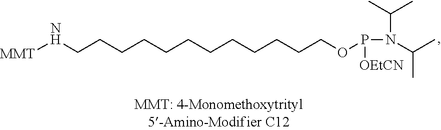

5′-thiol-modifier C6 of the structure:

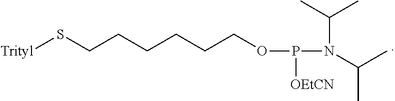

Polyethylene glycols (PEGs) can be conjugated to biologically active compounds to serve as “inert” carriers to potentially (1) prolong the half-life of the compound in the circulation, (2) alter the pattern of distribution of the compound and/or (3) camouflage the compound, thereby reducing its immunogenic potential and protecting it from enzymatic degradation. PEGs can range in size from 5 to 200 KD, with typical PEGs used in pharmaceutical formulations in the 10-60 KD range. Linear chain PEGs of up to about 30 KD can be produced. For PEGs of greater than 30 KD, multiple PEGs can be attached together (multi-arm or ‘branched’ PEGs) to produce PEGs of the desired size. The general synthesis of compounds with a branched, “mPEG2” attachment (two mPEGs linked via an amino acid) is described in Monfardini, et al. (1995) Bioconjugate Chem. 6:62-69. For ‘branched’ PEGs, i.e. compounds that include more than one PEG or mPEG linked to a common reactive group, the PEGs or mPEGS can be linked together through an amino acid such as a lysine or they can be linked via, for example, a glycerine. For branched PEGs in which each mPEG is about 10, about 20, or about 30 KD, the total mass is about 20, about 40 or about 60 KD and the compound is referred to by its total mass (i.e. 40 kD mPEG2 is two linked 20 kD mPEGs). 40 KD total molecular weight PEGs, that can be used as reagents in producing a PEGylated compound, include but are not limited to, for example, [N²-(monomethoxy 20K polyethylene glycol carbamoyl)-N⁶-(monomethoxy 20K polyethylene glycol carbamoyl)]-lysine N-hydroxysuccinimide of the structures:

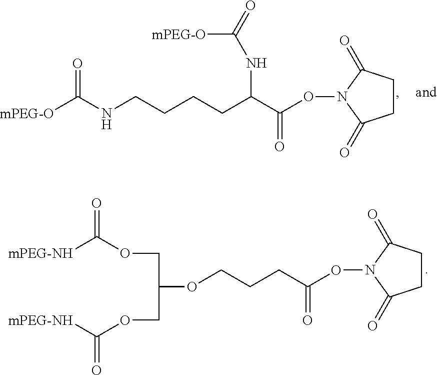

Additional PEG reagents that can be used to prepare stabilized compounds include other branched PEG N-Hydroxysuccinimide (mPEG-NHS) of the general formula:

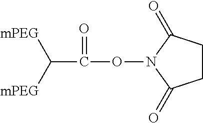

with a 40 kD or 60 kD total molecular weight (where each mPEG is about 20 or about 30 kD).

PEG reagents for the described compounds may include non-branched mPEG-Succinimidyl Propionate (mPEG-SPA), of the general formula:

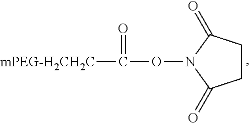

in which mPEG is about 20 kD or about 30 kD. In a specific embodiment, the reactive ester is —O—CH₂CH₂—CO₂—NHS.

Additional PEG reagents include a branched PEG linked through glycerol:

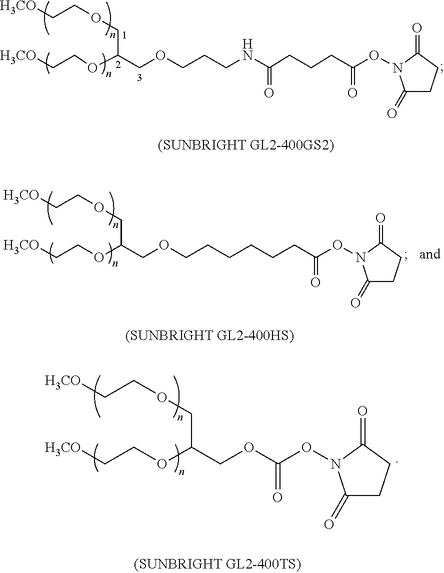

non-branched Succinimidyl alpha-methylbutanoate (mPEG-SMB) of the general formula:

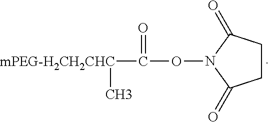

nitrophenyl carbonate linked PEGs, such as of the following structure:

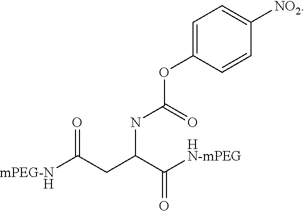

PEGs with thiol-reactive groups that can be used with a thiol-modified linker include compounds of the general structure:

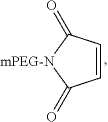

in which mPEG is about 10, about 20 or about 30 kD. Additionally, the structure can be branched, such as

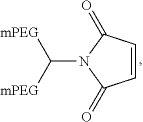

in which each mPEG is about 10, about 20, or about 30 kD and the total mass is about 20, about 40, or about 60 kD.

Branched PEGs may also include compounds of the general structure:

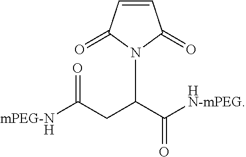

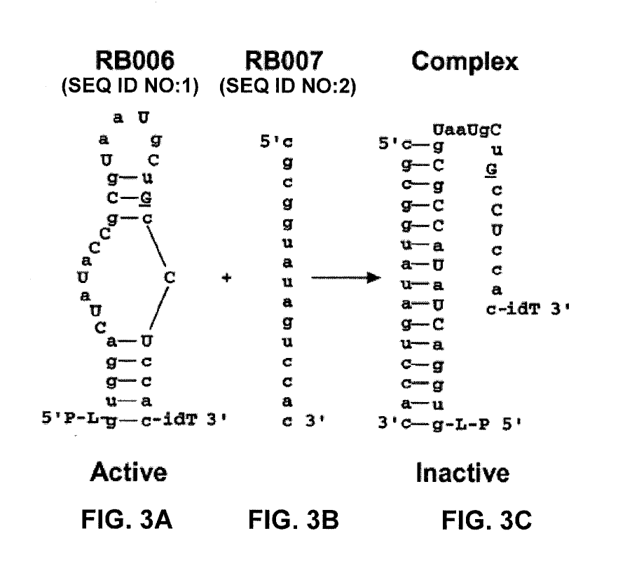

Described below as an example of an improved process for the manufacturing of a pegylated oligonucleotide is a description of alternate processes for the manufacture of a pegylated aptamer product known as RB006. RB006 refers to an oligonucleotide aptamer: P-L-guggaCUaUaCCgCgUaaUgCuGcCUccacT wherein P=mPEG2—NHS ester MW 40 kDa; L=C6 NH₂ linker; G=2-OH G; g=2′-O-Me G; C=2-F C; c=2′-O-Me C; U=2-F U; u=2′-O-Me U; a=2-O-Me A; and T=inverted 2′-H T (see FIG. 3A, SEQ ID NO:1). RB006 is the drug component of REG1, and is a direct FIXa inhibitor that binds coagulation factor IXa with high affinity and specificity (see U.S. Pat. No. 7,304,041 and Dyke et al., Circulation, 114:2490-97 (2006)). RB006 elicits an anticoagulant effect by blocking the FVIIIa/FIXa-catalyzed conversion of FX to FXa. RB006 is a modified RNA aptamer, 31 nucleotides in length, which is stabilized against endonuclease degradation by the presence of 2′-fluoro and 2′-O-methyl sugar-containing residues, and stabilized against exonuclease degradation by a 3′ inverted deoxythymidine cap. The nucleic acid portion of the aptamer is conjugated to a 40-kilodalton polyethylene glycol (PEG) carrier to enhance its blood half-life.

An advantageous feature of RB006 is the ability to reverse its in vivo effects by administration of an active control agent, which can be complementary to at least a portion of the aptamer. For the purposes of the present disclosure, an active control agent for RB006 may be RB007, which is shown in FIG. 3B and has the (5′-3′) sequence: cgcgguauaguccac (SEQ ID NO:2). Other possible active control agents may be oligonucleotide (5′-3′) sequences comprising: cgcgguauaguccccau (SEQ ID NO:3); cgcgguauaguccc (SEQ ID NO:4), cgcgguauaguccauc (SEQ ID NO:5), cgcgguauagucag (SEQ ID NO:6), cgcgguauagucagg (SEQ ID NO:7), cgcgguauagucagag (SEQ ID NO:8), or cgcgguauaguccucac (SEQ ID NO:9), or any modification or derivative thereof. In certain embodiments, the active control agent consists essentially of one of the above sequences, or consists entirely of one of the above sequences.

RB007 can effectively bind to RB006, thereby neutralizing its anti-FIXa activity. In one embodiment, 2′-O-methyl modification of RB007 confers moderate nuclease resistance to the molecule, which provides sufficient in vivo stability to enable it to seek and bind RB006, but does not support extended in vivo persistence.

It is understood that the presently described methods of manufacturing may be applied to any oligonucleotide or aptamer. Examples of additional aptamers and oligonucleotides which, in pegylated form, may be prepared using the methods disclosed herein are described below in Tables 1 and 2.

TABLE 1
Aptamers for Functional Regulation

| RB ID | Modified Sequence |
|---|---|
| RB448 | rGrGrGrArGrGrAfCrGrGfCrGfCfUrArAfCrGfCfCfUrGrGfCrAfUrArArGfCfCfUfCfCfCiT |
| RB450 | mGmGrGrArGrGrAfCrGrGfCrGfCfUrArAfCrGfCfCfUrGrGfCrAfUrArArGfCfCfUfCmCmCiT |
| RB451 | mGmGmGrArGrGrAfCrGrGfCrGfCfUrArAfCrGfCfCfUrGrGfCrAfUrArArGfCfCfUmCmCmCiT |
| RB452 | mGmGmGmArGrGrAfCrGrGfCrGfCfUrArAfCrGfCfCfUrGrGfCrAfUrArArGfCfCmUmCmCmCiT |
| RB566 | rGmGmAmGmGAfCG(s)G(s)mCmG(6GLY)mCmGfCfCfUmGmGfCmAfUmAmAmGmCmCmUmCmCiT |
| RB567 | (C6L)mGmGmAmGmGAfCG(s)G(s)mCmG(6GLY)mCmGfCfCfUmGmGfCmAfUmAmAmGmCmCmUmCmCiT |
| RB569 | (PEG40KGL2-NOF)(C6L)mGmGmAmGmGAfCG(s)GmCmG(6GLY)mCmGfCfCfUmGmGfCmAfUmAmAmGmCmCmUmCmCiT |
| RB570 | (PEG40KGL2-NOF)(C6L)mGmGmAmGmGAfCGGmCmG(6GLY)mCmGfCfCfUmGmGfCmAfUmAmAmGmCmCmUmCmCiT |
| RB571 | (PEG40KGL2-NOF)(C6L)mGmGmAmGmGAfCG(s)G(s)mCmG(6GLY)mCmGfCfCfUmGmGfCmAfUmAmAmGmCmC |
| | mUmCmCiT |

All ligands described in the column titled, “Modified Sequence” are modified versions of SEQ ID NO: 10 (RB448) rG = 2′Ribo G; rA = 2′Ribo A; mG = 2′O-Methyl G; mA = 2′O-Methyl A; mC = 2′O-Methyl C; mU = 2′O-Methyl U; fC = 2′Fluoro C; fU = 2′Fluoro U; fG = 2′Fluoro G; fA = 2′Fluoro A; iT-inverted deoxythymidine; (s)-phosphorothioate linkage; (C6L) = hexylamino linker; (6GLY) = hexaethylene glycol linker ((9-O-Dimethoxytrityl-triethylene glyco1,1-[(2-cyanoethyl)-(N,N-diisopropyl)]-phosphoramidite); (PEG40KGL2-NOF) = 40 kDa Branched PEG (SUNBRIGHT GL2-400GS2 product);
The “RB ID” is a unique identifier that refers to the aptamer having the sequence with specific modifications noted in the column titled, “Modified Sequence.”
The “SEQ ID NO:” refers to the corresponding nucleic acid sequence (DNA and/or RNA) without modifications.

TABLE 2
Modulator Oligonucleotides

| Name | RB ID | Modified Sequence | SEQ ID NO |
|---|---|---|---|
| EF-3 CA3 | RB422 | mUmUmAmCmGmCmAmAmGmAmCmGmCmGmGmU | 12 |
| EF-3 CA4 | RB423 | mUmGmUmGmAmUmCmCmGmCmAmUmCmGmUmC | 13 |
| RB490 CA 1 | RB513 | mGmGmGmAmGmGmCmUmUmAmUmGmCmCmAmGmGmCmG | 14 |
| RB490 CA 2 | RB514 | mGmAmGmGmCmUmUmAmUmGmCmCmAmGmGmCmG | 15 |
| RB490 CA 3 | RB515 | mUmUmAmUmGmCmCmAmGmGmCmG | 16 |
| RB490 CA 4 | RB516 | mCmGmCmCmGmUmCmCmUmCmCmC | 17 |
| RB538/571 | RB543 | mGmCmUmUmAmUmGmCmCmAmGmGmCmG | 18 |
| Control Agent 5 | | | |
| (14mer) | | | |
| RB538/571 | RB544 | mGmGmCmUmUmAmUmGmCmCmAmGmGmCmG | 19 |
| Control Agent 6 | | | |
| (15mer) | | | |
| RB538/571 | RB545 | mAmGmGmCmUmUmAmUmGmCmCmAmGmGmCmG | 20 |
| Control Agent 7 | | | |
| (16mer) | | | |

SEQ ID NOs. 12-20 correspond to the unmodified versions of the modulators described in the column titled “Modified Sequence.”

Further teachings related to RB006, RB007, aptamers which bind and modulate GPVI, and other therapeutic aptamers can be found in U.S. Pat. Nos. 7,300,922; 7,304,041, 7,312,325; and 7,531,524, and U.S. Patent Publication No. 2010/0311820, the contents of which are incorporated herein by reference in their entirety.

Two methods for the manufacture of RB006 are described herein for illustrative purposes, but are not meant to be limiting in scope as it will be apparent to those skilled in the art that various changes and modifications can be made without departing from the invention. Moreover, the methods described below can be used for other therapeutic aptamers as well as oligonucleotides which bind the aptamers, as described above. The two methods of manufacture are referred to as Process 1 and Process 2 and are described in detail below.

Table 3 lists the raw materials used in the manufacture of RB006 with Process 1 and Process 2. Any substitutions are considered minor as the chemical reactivity of the reagents is equivalent and no impact is expected on the quality of the API produced as a consequence of the change.

TABLE 3
Raw Materials Used in RB006 Manufacturing Process

| | Process 1 | Process 2 |
|---|---|---|
| mA(2′-O-Methyl-A^{Bz}) | Monomer | Monomer |
| mC(2′-O-Methyl-C^{Ac}) | Monomer | Monomer |
| mG(2′-O-Methyl-G^{iBu}) | Monomer | Monomer |
| mU(2′-O-Methyl-U) | Monomer | Monomer |
| fC(2′-F—C^{Ac}) | Monomer | Monomer |
| fU(2-′F—U) | Monomer | Monomer |
| rG(2′-O-TBDMS-G^{iBu}) | Monomer | Monomer |
| C6L(hexylamino linker) | Monomer | Monomer |
| PEG-NHS ester (40 kD PEG) | Monomer | Monomer |
| Inverted T Controlled Pore Glass (CPG) | Solid Support | Solid Support |

### Process 1.

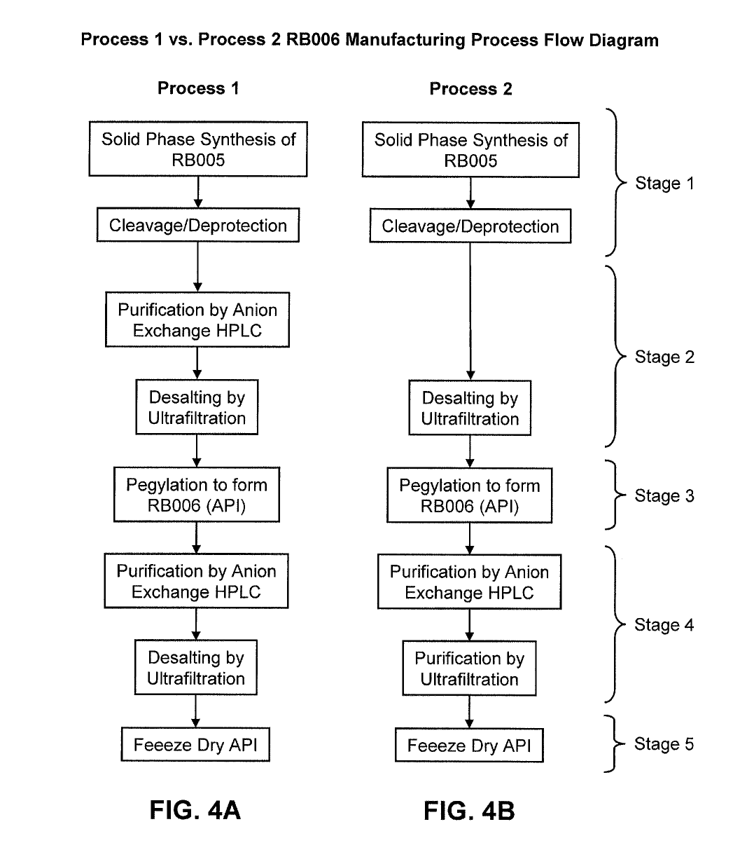

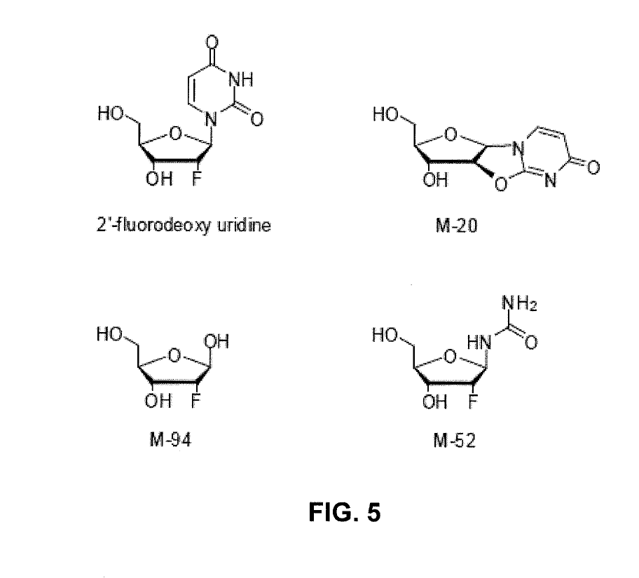

One method for the preparation of the pegylated oligonucleotide includes two anion exchange purification steps. This process, referred to herein as Process 1 and shown schematically in FIG. 4A, is described in five stages. Stage 1 is the synthesis on solid support and deprotection. Stage 2 encompasses the process of preparing the oligonucleotide for pegylation and includes purification by anion exchange HPLC. As the purification is performed using a sodium cation, the purification step also results in a salt exchange of ammonium and alkylammonium salts with sodium. The final step in the stage is desalting of the oligonucleotide prior to the pegylation reaction. Stage 3 comprises the pegylation and formation of the pegylated oligonucleotide. Stage 4 includes purification by anion exchange HPLC and desalting step prior to freeze drying in Stage 5.

### Process 1, Stage 1.

Stage 1 included solid phase synthesis of RB005 followed by cleavage and deprotection. During assembly of the 31-base oligonucleotide by solid phase synthesis, the last step of the coupling reaction is the addition of the hexylamino linker which provides the site specific attachment for pegylation (see FIG. 2A). The resulting full-length product with the hexylamino linker, prior to pegylation, is referred to as the nonPEGylated aptamer (RB005). The characterization of the crude oligonucleotide revealed two classes of impurities. The first is non-pegylatable due to lack of the hexylamino linker. The second is pegylatable and contains the hexylamino linker. Characterization of the pegylatable impurities revealed sequences which are shorter (N−1) and longer (N+1), both containing the hexylamino linker. In addition, pegylatable impurities with molecular weights closely related to the nonPEGylated aptamer were also detected. These impurities are designated as M−x and M+x, where M is the molecular weight of RB005 and x is the loss or gain in molecular weight. Two sets of experiments were performed in order to characterize impurities as nonpegylatable or pegylatable.

To confirm that non-linker containing oligonucleotides are non-pegylatable, experiments were performed with a version of nonPEGylated aptamer synthesized without the hexylamino linker. This nonlinker containing nonPEGylated aptamer was combined with mPEG2 NHS ester under standard reaction conditions and shown not to pegylate. To assess which impurities in the crude synthesis are pegylatable, a model study was performed in which the crude synthesis product and a surrogate for the mPEG2 NHS ester were reacted under standard pegylation conditions and the molecular weight of the products determined by high performance liquid chromatography mass spectrometry (LC-MS). The surrogate NHS ester has the same core structure as mPEG2 and is predicted to have the same reactivity. The same characterization cannot be performed with mPEG2 attached to RB005 (RB06) due to the polydispersity of the PEG and charge states from the oligonucleotide. The resulting characterization identified N−1, M−x, M+x, and N+1 as pegylatable species. Therefore, the focus of the characterization of the process has subsequently been on evaluating the levels of N−1, M−x, M+x, and N+1 species at various stages of manufacturing.

The characterization of the oligonucleotide suggested that further optimization of the synthesis cycle parameters could result in a lower level of N−1 and N+1 pegylatable impurities.

### Process 1, Stage 2.

The anion exchange purification step utilized prior to pegylation in Process 1 was expected to remove non-pegylatable and pegylatable oligonucleotide impurities and remove reagents from the deprotection step. However, extensive analysis of the pre and post purification samples surprisingly indicated that this purification step was only partially effective at removing oligonucleotide impurities impacting the final quality of the drug substance. Both LC-MS and chromatographic techniques were applied in the analysis.

The LC-MS method utilized provides semi-quantitative data on a relative ion count-% basis. Analysis by this LC-MS method confirmed that there was no meaningful difference in the purity of nonPEGylated aptamer as a result of anion exchange. The data presented in Table 4 show that there are slight shifts in the distribution of the impurities pre and post anion exchange purification but that the overall purity is comparable. There is an increase in M−x process related impurities following anion exchange purification and a corresponding decrease in the M+98, N−1 and N+1 impurities. A more detailed discussion of the M−x impurities is presented below

TABLE 4
LCMS comparison of purity and impurities in pre-
purified and purified RB005 (Ion current-%)

| | Change upon | Change upon |
|---|---|---|
| RB006 Batch | purification Lot 2 | purification Lot 3 |
| Use | Phase I Clinical | Phase I Clinical |
| | Other studies | |
| Sum N − 1 | −1.7 | −1.3 |
| Sum M − x | 1.9 | 4.2 |
| RB005 Purity | 3.3 | 1.3 |
| M + 98 | −2.7 | −2.9 |
| Sum N + 1 | −0.7 | −1.2 |

1) A positive number is an increase in purity or impurity level following purification. A negative number means the impurity level was decreased following purification

In summary, it appears that anion exchange HPLC after synthesis of nonPEGylated aptamer but before pegylation provided minimal improvement in the quality of nonPEGylated aptamer. Further, any meaningful improvement in the quality of the nonPEGylated aptamer taken forward to the pegylation step can most likely be achieved by continued improvement in the quality of synthesis and not by purification.

### Process 1, Stage 3.

In Process 1, two equivalents of mPEG2 NHS ester were used for each equivalent of nonPEGylated aptamer and the pegylation reaction was performed in an aqueous mixture of acetonitrile and sodium borate. While this reaction appeared to be adequate, a development effort was made to reduce the number of equivalents of this reagent required in the production of PEGylated aptamer (RB006) and thereby minimize the level of non-reacted mPEG2 to be removed during downstream processing. The pegylation rate is driven by pH, the solubility of the mPEG2 NHS ester, solubility of nonPEGylated aptamer, and the amount of water present in the reaction. Too much water results in hydrolysis of the reactive NHS ester, too little may result in precipitation of the oligonucleotide. Neither of these conditions impact the quality of drug substance but the efficiency of the pegylation reaction is sacrificed.

### Process 1, Stage 4.

The anion exchange purification of PEGylated aptamer used in Process 1 was evaluated for its ability to remove excess mPEG2 and non-pegylatable impurities. The non-anionic nature of mPEG2 allows the anion exchange column to effectively remove non-reactive mPEG2. Short nonpegylatable oligonucleotide impurities were removed during the anion exchange step of Stage 2. Non-pegylatable oligonucleotide impurities closely related to nonPEGylated aptamer, which weren't removed in Stage 2, are effectively removed in Stage 4 by preparative anion exchange purification, as the PEG moiety of the PEGylated aptamer results in the PEGylated aptamer eluting much earlier than the non-pegylatable oligonucleotide impurities closely related to the nonPEGylated aptamer.

Additional concentration of PEGylated aptamer was achieved by distillation of water in order to support the packaging requirements for freeze drying RB006 in vials.

### Process 1, Stage 5.

The physio-chemical characteristics of the PEGylated aptamer were assessed as part of the scale-up of the freeze dry process in order to determine appropriate shelf temperatures for freezing and primary drying.

### Process 2.

Based upon the characterization of various stages of Process 1, changes were made to increase the efficiency of the PEGylated aptamer manufacture as discussed below. A side-by-side comparison of Process 1 and Process 2 is provided in FIG. 4B.

The manufacturing process of Process 2 is again described in five stages. Stage 1 is the synthesis on solid support and deprotection. Stage 2 encompasses the process of preparing nonPEGylated aptamer for pegylation and includes the desalting of the product prior to the pegylation reaction by ultrafiltration. Stage 3 comprises the pegylation and formation of PEGylated aptamer. Stage 4 includes the purification by anion exchange HPLC and purification and desalting using ultrafiltration prior to the freeze-drying in Stage 5.

### Process 2, Stage 1.

In Process 1, synthesis of nonPEGylated aptamer was performed at 3 mmol scale. Material requirements for clinical studies and commercial feasibility necessitated increasing the scale in Process 2. Synthesis in Process 2 was performed at 10 and 20 mmol and 250 mmol scales and additional scale-up is possible through the course of development, such as at 300 mmol, 350 mmol, or 400 mmol scale. A larger scale synthesizer was utilized for Process 2. The chemical route of synthesis remained un-changed along with the starting monomers. Purity at the synthesis stage increased compared to that achieved with Process 1. Table 5 summarizes the scale of synthesis, purity by anion HPLC and yield of full-length product (FLP) reported in ODs/mmol. The intended use and process of manufacturing is also indicated. The material from the scaled up procedure is comparable to the material produced at the lower scale. The crude quality was 6% higher for Lot #5 as compared to previous clinical lots.

TABLE 5
RB005 In-process synthesis comparison
from original toxicology lot

| | Process 1 | | | Process 2 | |
|---|---|---|---|---|---|
| | Tox | Clinical | Clinical | Toxicology | Clinical |
| | Lot 1 | Lot 2 | Lot 3 | Lot 4 | Lot 5 |
| Synthesis | 3 | 3 | 3 | 20 | 10 |
| Scale | | | | | |
| (mmol) | | | | | |
| Change | NA | 12%** | 12%*** | 13% | 18% |
| in purity | | | | | |
| by AX | | | | | |
| HPLC | | | | | |
| Change | NA | No change | 15% | 29% | 28% |
| in Yield | | | | | |
| (ODs/ | | | | | |
| mmol) | | | | | |

**Average of four 3 mmol syntheses independently determined
***Average of seven 3 mmol syntheses independently determined

### Process 2, Stage 2

In Process 1, anion exchange chromatography was used to remove non-pegylatable impurities and to exchange the amine salts from the deprotection reaction with sodium salts. In Process 2, non-pegylatable impurities were removed in Stage 4 through a combination of anion exchange and ultrafiltration. In Table 6, the two processes for removing nonpegylatable impurities are compared.

TABLE 6
Purification Process Comparison

| | Process 1 | Process 2 |
|---|---|---|
| Stage 2 | Removal of non-pegylatable | Salt exchange by |
| | impurities and salt exchange | Diafiltration |
| | by anion exchange HPLC | Desalting by |
| | Desalting by Diafiltration | Diafiltration |
| Stage 3 | Pegylation | Pegylation |
| Stage 4 | Removal of non-reactive | Removal of non-reactive |
| | mPEG2, non-pegylatable | mPEG2, non-pegylatable |
| | impurities and | impurities and |
| | purification of RB006 | purification of RB006 |
| | using anion exchange HPLC | using anion exchange |
| | | HPLC and ultrafiltration |

Ultrafiltration is carried out by passing a solution of the oligonucleotide through an ultrafiltration membrane, whereby lower molecular weight impurities, such as ammonium and alkyl ammonium salts and solvents which may be present as residues from the synthesis and cleavage and deprotection, pass through the membrane, with the oligonucleotide being retained without passing through the membrane. The ultrafiltration serves to reduce the ratio of lower molecular weight impurities to oligonucleotide. The ultrafiltration can be operated in such a way so as to increase the concentration of the oligonucleotide in solution, by not adding fresh solvent (commonly water) to replace the volume passing through the ultrafiltration membrane, or by adding less water than the volume passed through. Alternatively, an equivalent or greater volume of solvent than that passed through the membrane can be added. The solution is commonly forced through the ultrafiltration membrane by the use of increased pressure.

The ultrafiltration step may be carried out in the presence of an aqueous sodium salt solution, such as a sodium chloride solution, in order to effect formation of the oligonucleotide in the desired sodium salt form, and to displace any residual ammonium, including alkylammonium, ions.

A plurality of ultrafiltration steps may be carried out if desired. In some embodiments, the ultrafiltration is performed in the presence of purified water, particularly following an ultrafiltration step in the presence of sodium salts. Ultrafiltration with purified water is commonly carried out until the oligonucleotide is substantially free from ammonium and residual inorganic sodium salts.

Concentrations of residual ions are often monitored by conductivity measurement, with values of less than 75 μS/cm, less than 50 μS/cm or less than 40 μS/cm, or ranging from about 20 μS/cm to about 50 μS/cm in the final permeate. In another embodiment, the final osmolality of the final permeate is less than or equal to about 4 mOsm, less than or equal to about 2 mOsm, less than or equal to about 1 mOsm, ranges from about 0.001 to about 1.0 mOsm, from about 0.5 mOsm to about 2.0 mOsm, or from about 0.5 mOsm to about 4.0 mOsm.

Ultrafiltration membranes used in the above-described processes can have a molecular weight cut-off selected to be lower than the molecular weight of the oligonucleotide. In embodiments where it is desired to remove alkylammonium ions from the solution, a molecular weight cut off higher than that of the alkylammonium ions may be employed. In many embodiments, for example with a typical 15 to 40 mer oligonucleotide having a molecular weight of approximately 4.5 to 12 kD, a molecular weight cut off in the range of from 1 kD to 3 kD is employed.

The process according to the present invention is often carried out at a temperature in the range of from 0° C. to about 50° C., or at ambient temperature, such as from about 15° C. to about 30° C.

The rationale for performing anion exchange purification of the non-pegylatable impurities in Stage 4 of Process 2 is driven by the specificity of the reaction of the mPEG2 NHS ester with the hexylamino linker. Improvements to the initial quality of synthesis and a reduction in the M−x impurity are supported by LC-MS analysis. Process 2 maintains the overall quality of nonPEGylated aptamer as compared to Process 1.

The data generated for in-process nonPEGylated aptamer are summarized in Table 7. The molecular weight for nonPEGylated is consistent with the theoretical molecular weight and the observed value is consistent for both processes. The sequencing data indicates that the Process 2 does not impact the expected sequence. The purity of the material produced from Process 1 and Process 2 are not directly comparable at this stage as the impurities removed in the anion exchange step of Process 1 are present in Process 2. In Process 2, the impurities are removed by the end of Stage 4.

TABLE 7
Batch History nonPEGylated aptamer
(In-Process) changes from Lot 1.

| | Batch History RB005 (In-Process) | | | | |
|---|---|---|---|---|---|
| | Lot 1 | Lot 2 | Lot 3 | Lot 4 | Lot 5 |
| Process | Process 1 | Process 1 | Process 1 | Process 2 | Process 2 |
| Change in | NA | 11% | 11% | 12% | 18% |
| crude purity | | | | | |
| from Lot 1 | | | | | |
| by AX- | | | | | |
| HPLC | | | | | |
| Change in | NA | −1% | −2% | NA* | NA* |
| Post- | | | | | |
| Purification | | | | | |
| Purity from | | | | | |
| Lot 1 by | | | | | |
| AX-HPLC | | | | | |

*NA: Not Applicable-A direct comparison can not be made at this stage as the material is not purified

As discussed above, only impurities containing the hexylamino linker are pegylatable and the anion exchange purification step in Process 1 had a minimal impact on removing these impurities. LC-MS analysis was used to identify and compare the pegylatable impurities present in material produced by Process 1 and Process 2 at this stage. The LC-MS method utilized provides semi-quantitative data on a relative ion count-% basis. The LC-MS data showed that the same types of impurities are expected in the pegylated product from Process 1 and Process 2. In Table 8 the calculated masses and tentative identities for pegylatable impurities are shown along with the relative impurity levels for both Process 1 and Process 2. Table 8 summarizes the relative N−1, M−x, nonPEGylated aptamer, M+x, and N+1 species from Process 1 and Process 2. The nomenclature M−x and M+x refers to impurities that are closely related in mass to nonPEGylated aptamer wherein M is the mass of nonPEGylated aptamer and x is the loss or gain of mass. The nomenclature N+1 and N−1 are those species either having one additional nucleotide present or missing a nucleotide. Since the non-pegylatable impurities have been demonstrated not to react, they are not included in the analysis and the data is normalized with respect to nonPEGylated aptamer.

TABLE 8
Change in Summed LC-MS data for in-process nonPEGylated
aptamer from Process 2 to Process 1 (Ion Current %)

| | Change from Process 1* | |
|---|---|---|
| RB006 Batch | Lot 4 | Lot 5 |
| Sum N − 1 | −0.5 | 0.3 |
| Sum M − x | −2.6 | −3 |
| RB005 Purity | 2.2 | 2.5 |
| M + 98 | 0 | 0 |
| Sum N + 1 | 0.9 | 0.3 |

*A positive number is an increase in the purity or impurity from the average values observed in Process 1. A negative value represents a decrease impurity level in Process 2 compared to the average values observed in Process 1.

### Impurities.

In both processes N+1 and N−1 impurities were observed containing the hexylamino linker. The N+1 family of impurities result from double coupling and the N−1 family from internal deletions. The M−94, M−52, and M−20 species are consistent with modification of 2′-deoxy-2′-fluoro uridine occurring during the manufacturing process. Heat and solutions with basic pH are shown to increase the level of these impurities. The molecular weights of the process related impurities (M−x) are consistent with the following structures:

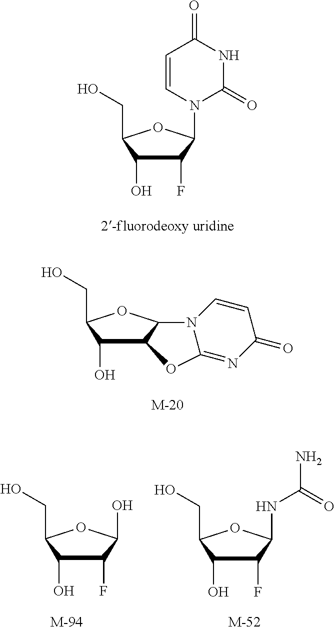

The species are consistent with degradation of 2′-deoxy-2′-fluoro uridine.

In Process 1, the impurity, M+98, is consistent with an impurity related to the linker starting material. The starting material impurity was tentatively identified to be a bi-functional hexylamino linker phosphoramidite. In Process 1, this impurity was removed by preparative anion exchange purification prior to pegylation. In Process 2, a gas chromatographic/FID QC method has since been implemented for control of this impurity in the starting material. This impurity is well controlled at the raw material stage and is not present in any of the batches produced by Process 2.

TABLE 9
Summary of Pegylatable Impurities and Purity
of by AX-HPLC (Area-%) from Lot 2

| | Process 1 | | Process 2 | |
|---|---|---|---|---|
| RB006 Batch | Purified Lot 2 | Purified Lot 3 | Lot 4 | Lot 5 |
| Tentative | | | | |
| Identification | | | | |
| Difference in | NA | −0.6% | +1.1% | −0.8% |
| Potential Pegylatable | | | | |
| impurities | | | | |
| RRT 0.91-0.99 and | | | | |
| 1.01-1.15 | | | | |
| Change in Purity by | NA | +0.6% | −0.9% | +0.8% |
| AX-HPLC | | | | |

TABLE 10
Summary of Pegylatable Impurities and
Purity by IP-HPLC (Area-%) from Lot 2

| | Process 1 | | Process 2 | |
|---|---|---|---|---|
| RB006 Batch | Lot 2 | Lot 3 | Lot 4 | Lot 5 |
| Tentative | | | | |
| Identification | | | | |
| Difference in | NA | −2.9% | −2.6% | −3.3% |
| Potential | | | | |
| pegylatable Sum | | | | |
| RRT 0.95-0.99 and | | | | |
| RRT 1.01-1.17 | | | | |
| Change in Purity | | +3.1% | +2.7% | +3.3% |
| By IP-HPLC | | | | |

In summary, the LC-MS and chromatographic data taken together show there is a decrease in the M−x pegylatable impurities resulting from the purification process and a minimal increase in N−1 and N+1 when the first anion exchange purification step is removed. Batch analysis by LC-MS is presented in Table 8. Similarly, all the impurities present in the clinical batch produced by Process 2 are present at equivalent or lower levels than in the earlier batches. The overall purity by LC-MS for material produced from Process 2 is better than for Process 1.

In Process 1 anion exchange purification was used to perform a salt exchange and remove primary amines from crude nonPEGylated aptamer during the purification step. This is an important operation as the conjugation will not proceed in the presence of amine salts. The ultrafiltration was performed using a 1 kD molecular weight cutoff (MWCO) membrane. In Process 2, since the removal of non-pegylatable impurities is moved to Stage 4, the ultrafiltration step was modified to include diafiltration against sodium chloride to achieve the salt exchange required for efficient PEG conjugation. In addition, the 1 kD membrane was replaced with a 5 kD MWCO membrane to improve flux rate.

### Process 2, Stage 3.

In Process 1, two equivalents of PEG-NHS ester were used for each equivalent of nonPEGylated aptamer and the pegylation reaction was performed in a mixture of acetonitrile and sodium borate. A development effort was made to reduce the number of equivalents of this reagent required in the production of PEGylated aptamer. A variety of conditions were evaluated for the pegylation that included reaction time, temperature, pH, PEG-NHS equivalents and solvent. The results of the study suggested that while the pegylation efficiency varied with the different conditions, the impurity profile of the pegylated product produced remained consistent. In Process 2, 1.5-1.75 equivalents of PEG-NHS ester are used in a mixture of ACN:DMSO and sodium borate buffer to produce PEGylated aptamer. Specificity of the new pegylation conditions were evaluated with non linker containing nonPEGylated aptamer and the mPEG2 NHS ester. As previously discussed, the aptamer without the linker was non reactive under the pegylation conditions. Comparison of in-process material produced by Process 2 to Process 1 is complicated by the presence of non-pegylatable impurities that are present in Process 2. The non-pegylatable impurities are removed in Stage 4. Therefore, a direct comparison of material produced using the two processes is not possible at Stage 3. Consequently, comparison of the quality of the material produced by both processes is accomplished by review of the data presented in the Batch History, Table 10 and the additional analytical characterization as described above.

### Process 2, Stage 4.

In manufacturing Process 1, the preparative anion exchange HPLC conditions for the Stage 4 purification were similar to those employed during Stage 2. In manufacturing Process 2, the purification conditions at Stage 4 were optimized from those used in Process 1; the change consisted of a modification to the gradient for purification and loading of the reaction mixture resulting from Stage 3. Comparison of in-process material produced by Process 2 to Process 1 is complicated by the presence of non-pegylatable impurities, and thus a direct comparison for material produced using the two processes was not performed.

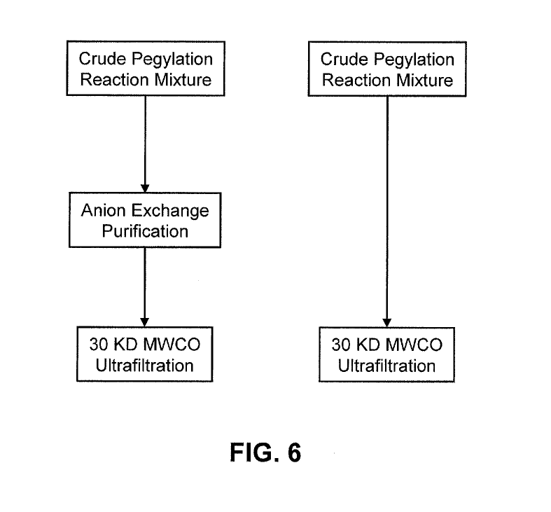

In manufacturing Process 1, ultrafiltration after pegylation was performed using a 5 kD MWCO membrane. The primary role of the ultrafiltration step in Process 1 was to desalt and concentrate RB006 prior to Freeze Dry. In Process 2, the MWCO membrane was changed to 10 or 30 kD to ensure that all non-pegylated oligonucleotides are removed prior to freeze dry. In addition, this increase in the molecular weight cut-off has the added benefit of improving the flux rate during diafiltration. An important element in developing manufacturing Process 2 was evaluating the process capability for removing non-pegylatable impurities. Although the majority of these impurities are removed in anion exchange purification of PEGylated aptamer, there is a possibility that non-pegylated oligonucleotides co-elute with the pegylated product and could be carried through to the final API. To test the extent that the 30 kD MWCO membrane was capable of removing non-pegylatable impurities in the range of 2 kD-10 kD, the pegylation reaction mixture from Stage 3 was loaded onto the 30 kD MWCO membrane without being subjected to the normal purification process. A schematic representation for the evaluation process relative to Process 2 is shown in FIG. 6.

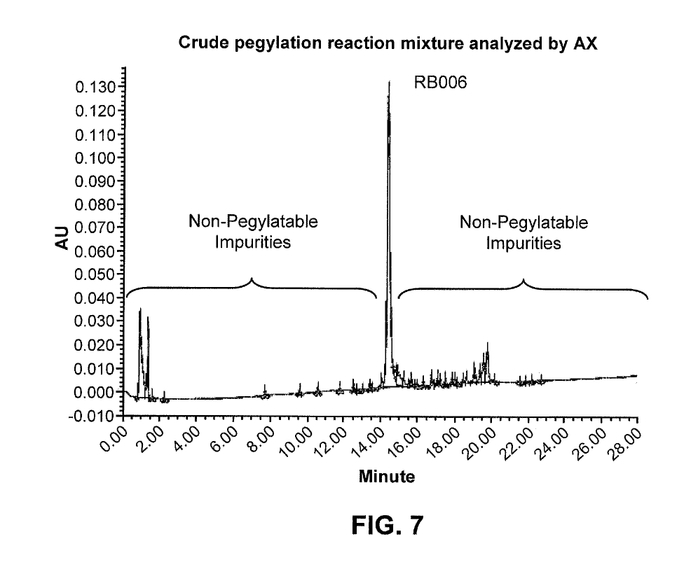

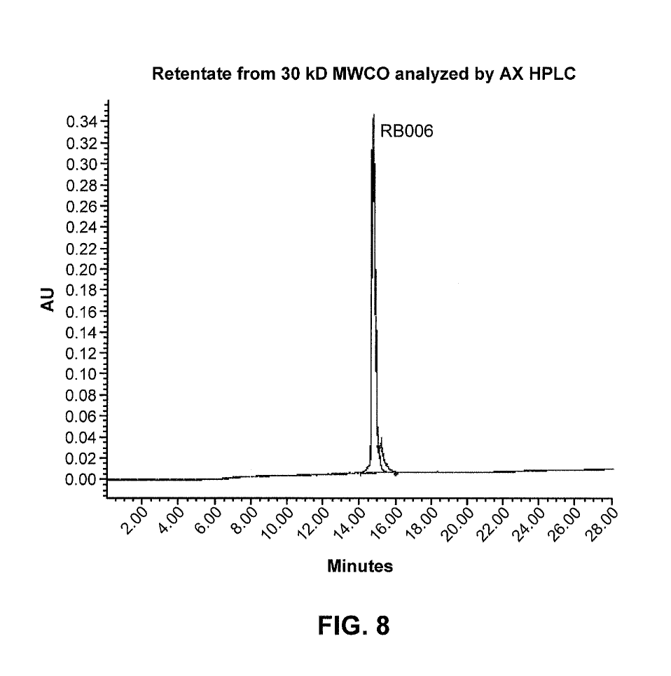

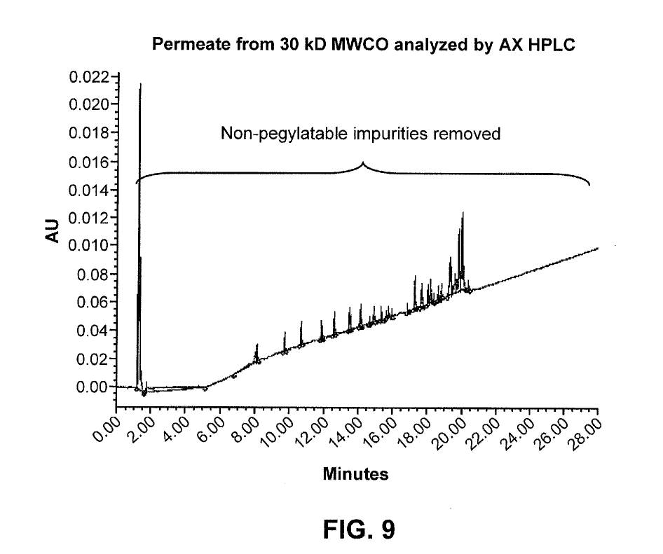

AX-HPLC (anion exchange HPLC) was used to analyze the pegylation reaction mixture, the UF permeate solution and UF retentate solution. The results are shown in FIG. 7-FIG. 9. The analytical HPLC of the material following ultrafiltration clearly demonstrates the ability of the process to remove non-pegylatable impurities, including those species that co-elute with PEGylated aptamer. In FIG. 7, the non-purified pegylation reaction mixture is shown. The retention time of PEGylated aptamer is 14.3 minutes. The retention times of 19-20 minutes correspond to nonpegylatable impurities closely related to the full-length product (N−1, N−2 . . . without linker) with an approximate molecular weight of 10,000 kD. In FIG. 8, the analysis of the retentate indicates the removal of a multitude of species from the pegylation reaction mixture. The impurities with retention times of 19-20 minutes have been removed by the UF process. Additionally, In FIG. 9, the analysis of the permeate solution is shown. The permeate shows that non-pegylatable species with retention times similar to the PEGylated aptamer are also removed. IP-HPLC and AX-HPLC are used in combination to monitor the removal of non-pegylatable impurities during the process of ultrafiltration.

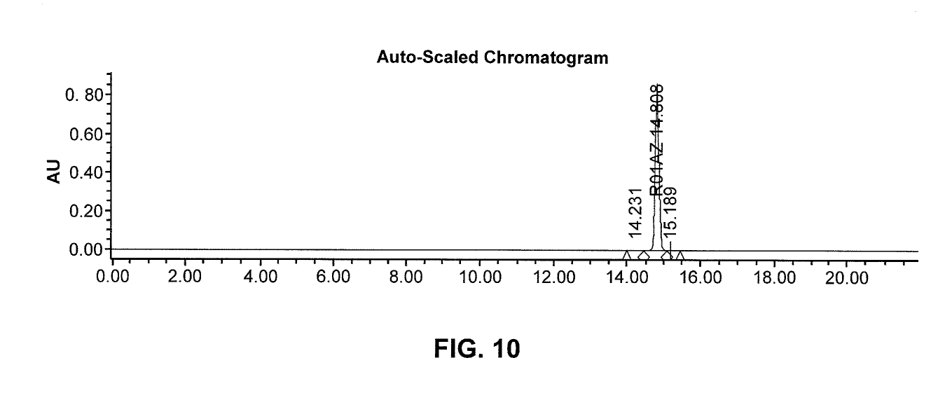

To evaluate the use of a 10 kD MWCO in the manufacturing process, pegylated oligonucleotide was purified by anion exchange HPLC (Stage 3 of the process) and then subjected to ultrafiltration using a 10 kD membrane. The material was then analyzed by analytical IP-HPLC, which is selective for non-PEGylated and pegylated oligonucleotide. The non-pegylatable oligonucleotides would be detected in between 2 and 6 minutes. FIG. 10 shows that no non-pegylatable oligonucleotides are present and that the process of utilizing an HPLC purification and a 10 kD membrane removes non-pegylatable oligonucleotides.

The purity of material produced by Process 2 at the end of Stage 4 is comparable to the purity of material produced by Process 1.

### Process 2, Stage 5.

In Process 1, PEGylated aptamer was freeze-dried in individual vials. In Process 2, the freeze-dry parameters were optimized based upon the physical characterization of RB006 and the material was freeze dried in bulk using lyoguard trays. The use of the lyoguard trays and the physio-chemical characterization allowed for PEGylated aptamer to be freeze dried at lower concentrations than necessary to support freeze drying in vials. The rationale for each change and the potential impact of the change on product quality are discussed. The changes were made to accommodate a more robust process, the potential for scale up, greater manufacturing flexibility and worker safety with little to no impact on product quality. Material produced using Process 1 and material produced using Process 2 met quality specifications, contained similar impurity profiles and were of similar purity overall. Changes made to all five stages of Process 1 to develop Process 2 allowed for increased ease and feasibility of scale up while maintaining or improving product quality.

TABLE 11
Process changes in the Manufacture of PEGylated
aptamer and Impact on Product Quality

| | | | | |
|---|---|---|---|---|
| Synthesis | | | | Impact on |
| Equipment | | | Reason for | Product |
| and Scale | Process 1 | Process 2 | Change | Quality |

Purification by AX or Desalting

| | | | | |
|---|---|---|---|---|
| Scale | 3 mmol | Anion Exchange | Purification was | None found |
| | | Purification was | ineffective in | |
| | | removed | improving N − 1 | |
| | | | and | |
| | | | N + 1 impurities | |
| Method | AX Resin | N/A | N/A | N/A |

Desalting by Ultrafiltration

| | | | | |
|---|---|---|---|---|
| UF cassettes | 1 KD Pall Maximate | 2 KD-5 KD | Improved Flux | None found |
| | (Polyethersulfone) | Sartorius | rate, | |
| | | Hydrostart | decreased | |
| | | (Regenerated | processing | |
| | | Cellulose) | time | |
| Reagents | WFI Quality Water | WFI Quality | Removal of | None found |
| | | Water. NaCl, | Amines | |
| | | WFI Quality Water | | |

Desalting by Ultrafiltration

| | | | | |
|---|---|---|---|---|
| UF cassettes | 5K Pall Maximate | 10-30 KD Pall | Purification Step | none found |
| | | Maximate | to | |
| | | | remove | |
| | | | nonpegylatable | |
| | | | impurities | |
| Reagents | WFI | No change | N/A | N/A |

Lyophilization

| | | | |
|---|---|---|---|
| Container | Glass vials (100 | Lyoguard Tray | Scale up |
| | mL) | | |

Methods include analysis by anion exchange HPLC, ion-pairing reverse-phase HPLC and two-column GPC. The improved anion exchange method utilizes a Tris buffer/acetonitrile/methanol/pH 8 system and a sodium chloride gradient, a Dionex DNAPac PA-100 (4×250 mm) column at 40° C. with a flow of 1.5 mL/min. Detection is at 259 nm. The method has been shown to resolve several synthetically prepared N+1 and N−1 pegylated impurities, RB006 cleavage products and RB006 which has been pegylated with a 20 kDa and 30 kDa species. The method is linear across the range of 50%-120% and 0.1%-10% of nominal concentration (2.0 mg/mL). The improved two-column GPC method uses Waters Ultrahydrogel 1000 and 500 (7.8×300 mm) columns in series and 20 mM ammonium acetate pH 7.4 at ambient temperature with isocratic elution at 0.4 mL/min. Detection is by evaporative light scattering (50° C. Drift tubes, 40 PSI Nitrogen, ESI cooling) and by UV at 259 nm. The method utilizes a series of polyethylene oxide and polyethylene glycol standards ranging from 72 kDa to 12 kDa for molecular weight determination. The method is linear across the mass range and the detection limit is 0.2%. The improved ion pair HPLC (IP-HPLC) analysis of RB006 is performed using a Waters Xbridge BEH300 C4 (4.6×100 mm, 3.5 μm) column using a gradient of mobile phase A: 100 mM triethylammonium acetate (TEAA), pH 7.2, 5% methanol and mobile phase B: 100 mM TEAA, pH 7.2, 50% acetonitrile, 30% isopropanol at pH 8.5 at a column temperature of 35° C. The flow rate is 1.0 mL/minute and detection is by UV at 259 nm.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the exemplary methods and materials are now described. All publications mentioned herein are incorporated herein by reference to disclose and describe the methods and/or materials in connection with which the publications are cited.

1. A method for preparing a therapeutic pegylated oligonucleotide comprising,
(a) synthesizing a non-pegylated oligonucleotide on a solid support,
(b) cleaving the non-pegylated oligonucleotide from the solid support and deprotecting the oligonucleotide,
(c) desalting the non-pegylated oligonucleotide using ultrafiltration,
(d) pegylating the non-pegylated oligonucleotide to produce a pegylated oligonucleotide,
(e) purifying the pegylated oligonucleotide using anion exchange HPLC, and
(f) desalting and further purifying the pegylated oligonucleotide using ultrafiltration,
wherein no ion-exchange purification of the non-pegylated oligonucleotide is performed between steps (b) and (c).

2. The method of claim 1, further comprising step (g) freeze drying the final product.

3. The method of claim 1, wherein the non-pegylated oligonucleotide comprises a secondary structure.

4. The method of claim 3, wherein the secondary structure comprises a stem and a loop.

5. The method of claim 1, wherein the non-pegylated oligonucleotide comprises a modified nucleotide.

6. The method of claim 5, wherein the non-pegylated oligonucleotide comprises a 2′-O-methyl modified nucleotide.

7. The method of claim 5, wherein the non-pegylated oligonucleotide comprises a 2′-fluoro modified nucleotide.

8. The method of claim 1, wherein the non-pegylated oligonucleotide is coupled to a linker prior to step d.

9. The method of claim 1, wherein the non-pegylated oligonucleotide comprises RB005.

10. The method of claim 1, wherein the pegylated oligonucleotide comprises RB006.

11. The method of claim 1, wherein step (f) comprises using an ultrafiltration membrane which has a molecular weight cutoff about 10 kD to about 20 kD or of about 20 kD to about 30 kD.


SEQUENCE LISTING (20 sequences)

SEQ ID NO: 1 - DNA, 31 bp, Artificial Sequence
  Synthetic aptamer
guggacuauaccgcguaaugcugccuccact

SEQ ID NO: 2 - RNA, 15 nt, Artificial Sequence
  Synthetic aptamer
cgcgguauaguccac

SEQ ID NO: 3 - RNA, 17 nt, Artificial Sequence
  Synthetic aptamer
cgcgguauaguccccau

SEQ ID NO: 4 - RNA, 14 nt, Artificial Sequence
  Synthetic aptamer
cgcgguauaguccc

SEQ ID NO: 5 - RNA, 16 nt, Artificial Sequence
  Synthetic aptamer
cgcgguauaguccauc

SEQ ID NO: 6 - RNA, 14 nt, Artificial Sequence
  Synthetic aptamer
cgcgguauagucag

SEQ ID NO: 7 - RNA, 15 nt, Artificial Sequence
  Synthetic aptamer
cgcgguauagucagg

SEQ ID NO: 8 - RNA, 16 nt, Artificial Sequence
  Synthetic aptamer
cgcgguauagucagag

SEQ ID NO: 9 - RNA, 17 nt, Artificial Sequence
  Synthetic aptamer
cgcgguauaguccucac

SEQ ID NO: 10 - RNA, 35 nt, Artificial sequence
  Synthetic Oligonucleotide RB448
gggaggacggcgcuaacgccuggcauaagccuccc

SEQ ID NO: 11 - RNA, 35 nt, Artificial sequence; identical to SEQ ID NO: 10
  Synthetic Oligonucleotide modified RB448

SEQ ID NO: 12 - RNA, 16 nt, Artificial sequence
  Synthetic Oligonucleotide EF-3 CA3
uuacgcaagacgcggu

SEQ ID NO: 13 - RNA, 16 nt, Artificial sequence
  Synthetic Oligonucleotide EF-3CA4
ugugauccgcaucguc

SEQ ID NO: 14 - RNA, 19 nt, Artificial sequence
  Synthetic Oligonucleotide RB490 CA1
gggaggcuuaugccaggcg

SEQ ID NO: 15 - RNA, 17 nt, Artificial sequence
  Synthetic Oligonucleotide RB490 CA2
gaggcuuaugccaggcg

SEQ ID NO: 16 - RNA, 12 nt, Artificial sequence
  Synthetic Oligonucleotide RB490 CA3
uuaugccaggcg

SEQ ID NO: 17 - RNA, 12 nt, Artificial sequence
  Synthetic Oligonucleotide RB490 CA4
cgccguccuccc

SEQ ID NO: 18 - RNA, 14 nt, Artificial sequence
  Synthetic Oligonucleotide RB538/571
gcuuaugccaggcg

SEQ ID NO: 19 - RNA, 15 nt, Artificial sequence
  Synthetic Oligonucleotide RB538/571
ggcuuaugccaggcg

SEQ ID NO: 20 - RNA, 16 nt, Artificial sequence
  Synthetic Oligonucleotide RB538/571
aggcuuaugccaggcg